(19) United States
(12) Patent Application Publication
(10) Pub. No.: US 2022/0160718 A1
(43) Pub. Date: May 26, 2022

(54) COMPOSITIONS AND METHODS OF TREATING CANCER

(71) Applicant: Bristol-Myers Squibb Company, Princeton, NJ (US)

(72) Inventors: Qihong Zhao, Princeton, NJ (US); Joseph Fargnoli, Pipersville, PA (US); Murali Gururajan, San Diego, CA (US); Susan Wee, Moorestown, NJ (US)

(21) Appl. No.: 17/581,481

(22) Filed: Jan. 21, 2022

Related U.S. Application Data
(63) Continuation of application No. 16/253,353, filed on Jan. 22, 2019, now abandoned.
(60) Provisional application No. 62/620,209, filed on Jan. 22, 2018.

(51) Int. Cl. A61K 31/53; C07K 16/28; A61P 35/00; A61K 39/395; A61P 35/04; A61K 9/00; A61K 45/06

(52) U.S. Cl. CPC A61K 31/53; C07K 16/2818; A61P 35/00; A61K 39/39541; A61K 2039/505; A61K 9/0019; A61K 45/06; C07K 16/2827; A61P 35/04

(57) ABSTRACT

The invention is directed to methods of treating cancer in subjects with a combination comprising a CCR2/5 dual antagonist, such as N-((1R,2S,5R)-5-(tert-butylamino)-2-((S)-3-(7-tert-butylpyrazolo[1,5-a][1,3,5]triazin-4-ylamino)-2-oxopyrrolidin-1-yl)cyclohexyl)acetamide; a monoclonal antibody, such as nivolumab; and/or chemotherapy.

### CROSS-REFERENCE TO RELATED APPLICATIONS

This application is a continuation of U.S. Ser. No. 16/253,353 filed Jan. 22, 2019, which claims the benefit of U.S. Provisional Application No. 62/620,209, filed Jan. 22, 2018, the disclosure of which is incorporated herein by reference in its entirety.

### TECHNICAL FIELD

This invention relates to methods of treating cancer in subjects with a combination of a CCR2/5 dual antagonist, a monoclonal antibody and/or chemotherapy. In some embodiments, the tumor is a solid tumor. In certain embodiments, the solid tumor is pancreatic cancer, colorectal cancer, or a combination thereof.

### BACKGROUND

Pancreatic adenocarcinoma is the third leading cause of cancer death in the United States and is projected to be the second leading cause of cancer death by 2020. The 5 year survival of pancreatic cancer is dismal with only 8% surviving 5 years from diagnosis. Outcomes in patients with metastatic disease is dismal with median survival of less than 1 year. Treatment options for patients with metastatic pancreatic cancer are limited. Gemcitabine combined with nab-paclitaxel is approved for 1L treatment for patients with advanced pancreatic cancers. Patients with advanced pancreatic cancer can be treated with 5-fluorouracil (5-FU)/liposomal irinotecan in the second-line (2L) setting. However, outcomes with these agents is generally poor with low overall response rate, a median PFS of only 2 to 3 months and median overall survival (OS) of 6 to 7 months. Although chemotherapy improves survival, there are significant toxicities and all patients eventually succumb to their disease. Development of new treatments for pancreatic cancer is an area of unmet need.

Worldwide, colon cancer (including rectal cancer) is the third most common form of cancer in men and second most common in women. In 2013 in the United States (US), an estimated 142,820 new cases of colon or rectal cancer (CRC) would be diagnosed, with an estimated 50,830 deaths due to CRC. At initial diagnosis, approximately 25% of patients present with metastatic disease and almost 50% of patients will develop metastasis which contributes to the high mortality rate reported in CRC patients. Treatment options for patients with metastatic colon or rectal cancer (mCRC) are predominantly 5-fluorouracil (5-FU) containing regimens in combination with either oxaliplatin or irinotecan (FOLFOX or FOLFIRI) with a biologic agent such as bevacizumab. The EGFR inhibitors, cetuximab and panitumumab, are also options if KRAS status is non-mutated. In later-line therapy, regorafenib, in patients who have been previously treated with chemotherapy has demonstrated an improvement in overall survival of about 6 months. Similar results in survival were demonstrated for triflurdine/tipiracil. Despite the numerous initial treatment options for mCRC, the benefit of these therapies is modest, and complete radiographical responses are rare, highlighting the need for more effective therapies.

Checkpoint inhibitors have transformed cancer care, but extending those benefits to more patients may require additional approaches. Cysteine-cysteine (C-C) chemokine receptor 2 (CCR2) and C-C chemokine receptor 5 (CCR5) are 2 chemokine receptors that are expressed on myeloid cell and T cell infiltrates in the tumor microenvironment (TME) and have been shown to be key drivers of the migration and accumulation of myeloid cells, including tumor-associated macrophages (TAMs) and myeloid-derived suppressor cells, into the TME (Mantovani, A. et al., Nat. Rev. Clin. Oncol., 2017 Jan. 24. doi: 10.1038/nrclinonc.2016.217; Lesokhin, A. M. et al., Cancer Res 72(4):876-86 (2012); Schlecker, E. et al., J. Immunol., 189(12):5602-11 (2012)). Moreover, both receptors have been shown to be important players in the trafficking of regulatory T cells (Treg) to the TME (Loyher, P. L. et al., Cancer Res., 76(22):6483-94 (2016); Tan M. C. et al., J. Immunol., 182(3):1746-55 (2009)). Besides its primary role in driving immune cell migration to the TME, CCR5 inhibition has been recently shown to repolarize TAMs from an immunosuppressive M2 phenotype to an immune-activated M1 phenotype (Halama, N. et al., Cancer Cell, 29(4):587-601(2016)). Each receptor has been separately shown to be an important player in multiple models of cancer, including pancreatic cancer (Sanford, D. E. et al., Clin. Cancer Res., 19(13):3404-15 (2013); Tan M. C. et al., J. Immunol., 182(3):1746-55 (2009)), colon cancer (Afik, R. et al., J. Exp. Med., 213(11):2315-31 (2016); Tanabe, Y. et al., 7(30):48335-45 (2016)), liver cancer (Li, X. et al., Gut, 66(1):157-67 (2017); Barashi, N. et al., Hepatology, 58(3):1021-30 (2013)) and lung cancer (Schmall, A. et al., Am. J. Respir. Crit. Care Med., 191(4):437-47 (2015); Lee, N.J. et al., Carcinogenesis, 33(12):2520-8 (2012)). CCR2-selective and CCR5-selective antagonists have shown positive proof of mechanism and clinical response in patients, in combination with chemotherapy, with pancreatic and colorectal cancer, respectively (Nywening, T. M. et al., Lancet Oncol., 17(5):651-62 (2016); Halama, N. et al., Cancer Cell 29(4):587-601 (2016)).

Use of a dual CCR2/5 antagonist in combination with an anti-PD-1 antibody and/or chemotherapy has not been reported and represents a novel approach with the potential to extend immuno-oncology benefits to patients not adequately served by existing therapies. Additionally, targeted therapy of multiple non-redundant molecular pathways regulating immune responses can enhance antitumor immunotherapy. There remains a need for combination therapies with an acceptable safety profile and high efficacy that enhance antitumor immune responses compared to monotherapy and other immunotherapy combinations.

### SUMMARY

The present invention is directed to, among other things, methods of treating cancer in a subject comprising administering to the subject a combination of a CCR2/5 dual antagonist, a monoclonal antibody, and/or chemotherapy.

In some embodiments, the cancer is a solid tumor. In certain embodiments, the cancer is colorectal cancer, pancreatic cancer, liver cancer and lung cancer, or a combination thereof. In certain embodiments, the cancer is colorectal cancer. In certain embodiments, the cancer is pancreatic cancer.

In some embodiments, the monoclonal antibody is an anti-PD-1 antibody. In some embodiments, the anti-PD-1 antibody cross-competes with nivolumab for binding to human PD-1. In some embodiments, the anti-PD-1 antibody binds to the same epitope as nivolumab. In certain embodiments, the anti-PD-1 antibody is nivolumab.

In some embodiments, the CCR2/5 dual antagonist is an equipotent dual antagonist of CCR2 and CCR5. In certain embodiments, the CCR2/5 dual antagonist is a compound of Formula (I), or a pharmaceutically acceptable salt thereof, or a combination thereof,

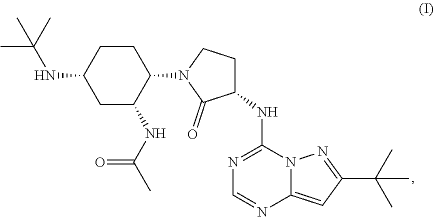 [SMILES: CC(=O)N[C@@H]1C[C@H](NC(C)(C)C)CC[C@@H]1N1CC[C@H](Nc2ncnc3cc(C(C)(C)C)nn23)C1=O]

N-((1R,2S,5R)-5-(tert-butylamino)-2-((S)-3-(7-tert-butylpyrazolo[1,5-a][1,3,5]triazin-4-ylamino)-2-oxopyrrolidin-1-yl)cyclohexyl)acetamide.

In some embodiments, the chemotherapeutic agents are selected from nab-paclitaxel, gemcitabine, 5-fluorouracil (5-FU), leucovorin, and irinotecan.

### BRIEF DESCRIPTION OF THE DRAWINGS

FIG. 1 illustrates synergistic combination of Compound A with Antibody B against mouse colon tumor (MC38) progression.

FIG. 2 illustrates synergistic combination of Compound A with Antibody B against mouse colon tumor (MC38) progression.

FIG. 3 illustrates synergistic combination of Compound A with Antibody B against mouse colon tumor (CT26) progression.

### DETAILED DESCRIPTION

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically carriers, and excludes other compounds.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

All ranges disclosed herein are inclusive of the recited endpoint and independently combinable (for example, the range of “from 200 mg to 600 mg” is inclusive of the endpoints, 200 mg and 600 mg, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 100 to about 200” also discloses the range “from 100 to 200.” The term “about” may refer to plus or minus 10% of the indicated number. For example, “about 10%” may indicate a range of 9% to 11%, and “about 1” may mean from 0.9 to 1.1. Other meanings of “about” may be apparent from the context, such as rounding off, so, for example “about 1” may also mean from 0.5 to 1.4.

CCR2/5 dual antagonist refers to a small-molecule antagonist that binds potently to CCR2 and CCR5 receptors and exhibits potent dual inhibition of in vitro receptor-mediated functions such as CCR2- and CCR5-mediated functions such as calcium flux and chemotaxis in response to their respective cognate ligands. Compounds having CCR2/5 dual inhibitory activity are reported in, for example, U.S. Pat. Nos. 8,383,812 and 7,163,937.

U.S. Pat. No. 7,163,937 (hereby incorporated by reference) discloses CCR2/5 dual antagonists including (S)-1-((1S,2R,4R)-4-(isopropyl(methyl)amino)-2-propylcyclohexyl)-3-((6-(trifluoromethyl)quinazolin-4-yl)amino)pyrrolidin-2-one (hereinafter referred to as Compound A). The structure of Compound A is

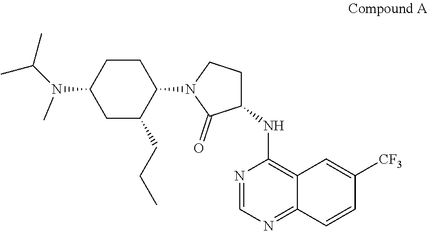 [SMILES: CCC[C@@H]1C[C@H](N(C)C(C)C)CC[C@@H]1N1CC[C@H](Nc2ncnc3ccc(C(F)(F)F)cc23)C1=O]

U.S. Pat. No. 8,383,812 (hereby incorporated by reference) discloses CCR2/5 dual antagonists including a compound of Formula (I):

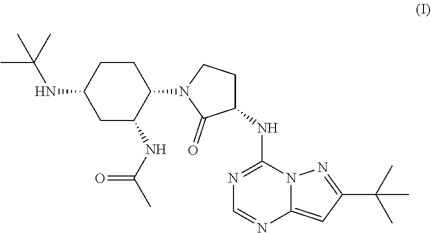 [SMILES: CC(=O)N[C@@H]1C[C@H](NC(C)(C)C)CC[C@@H]1N1CC[C@H](Nc2ncnc3cc(C(C)(C)C)nn23)C1=O]

N-((1R,2S,5R)-5-(tert-butylamino)-2-((S)-3-(7-tert-butylpyrazolo[1,5-a][1,3,5]triazin-4-ylamino)-2-oxopyrrolidin-1-yl)cyclohexyl)acetamide (hereinafter referred to as Compound C).

As shown, for example, Compound A potently blocks binding of CCL2 (also known as MCP-1), a ligand for CCR2, to mouse CCR2-expressing cells; potently blocks mouse CCL4 (also known as MIP-10), a ligand for CCR5, to mouse CCR5-expressing cells; potently inhibits mouse CCL2- and mouse CCL4-induced functions (calcium flux, integrin CD11b upregulation); and is pharmacologically related to Compound C (Table 1).

TABLE 1
Mouse CCR2/5 potency of Compound
A versus human potency of Compound C

| | Compound A Mouse | | Compound C Human | |
|---|---|---|---|---|
| | Pharmacology | | Pharmacology | |
| | CCR2 | CCR5 | CCR2 | CCR5 |
| | Potency | potency | Potency | Potency |
| Assay | (nM) | (nM) | (nM) | (nM) |
| Binding on | 0.8 | 0.4 | 6.2 | 3.6 |
| CCR2 or CCR5- | | | | |
| expressing cells | | | | |
| (displacing 125I- | | | | |
| labelled ligand) | | | | |
| Ligand-induced | 0.7 | 0.4 | 2.9 | 2.0 |
| calcium flux | | | | |
| in cells | | | | |
| Ligand-induced | 13.0 | 5.7 | 4.8 | 5.7 |
| integrin | | | | |
| upregulation in | | | | |
| whole blood | | | | |

### Assays for Inhibition of Ligand Binding to Cells Expressing CCR2 and to Cells Expressing CCR5

The human CCR2 and CCR5 binding assay was established with human peripheral blood mononuclear cells (hPBMCs) and human T cells using 125I-human MCP-1 and -human MIP-1beta as the tracer ligand, respectively, hPBMCs and human T cells were isolated from human leukopak using a standard protocols. Isolated cells (hPBMCs for CCR2 binding and human T cells for CCR5 binding) were washed and diluted to 1×107/ml in binding buffer (RPMI-1640, 0.1% BSA, 20 mM Hepes, pH 7.4). 125I-MCP-1 and -MIP-1beta (NEN/Perk Elmer) was diluted to 0.45 nM in binding buffer. Compound C was diluted in binding buffer at 3-fold the final concentrations used in the binding assay. The binding assay was performed using a 96-well filter plate (Millipore). Total 125I-MCP-1 and -MIP-1beta binding was assessed as follows: each reaction (150 μl) contained 5×105 cells, 0.15 nM 125I-MCP-1 or -MIP-1beta, and Compound C such that the final concentration ranged from 0 to 100 nM. The plate was incubated at room temperature for 30 minutes followed by three washes with RPMI-1640, 0.1% BSA, 0.4 M NaCl, 20 mM Hepes, pH 7.4 using a vacuum manifold filtration (Millipore). After washing, the plate was air-dried for 60 minutes at room temperature, followed by the addition of 25 μl of Microscint 20 into each well. The plate was sealed and counted on the Trilux scintillation counter for 1 minute. Non-specific binding was determined in the presence of 300 nM cold MCP-1 and MIP-1beta (PeproTech Inc.). Specific 125I-MCP-1 and -MIP-1beta binding was calculated as the difference between total and non-specific binding. All conditions were tested in duplicate. The IC50 is defined as the concentration of competing Compound C required to reduce specific binding by 50%.

Assays for inhibition of mouse ligand binding to mouse CCR2 and to CCR5 were established in similar fashion to the human assays except that WEHI-274.1 mouse monocyte line and L1.2 cells stably expressing mouse CCR5 were used as sources of mouse CCR2- and mouse CCR5-expressing cells, respectively, and mouse MCP-1 and -MIP-1beta were used as the tracer ligands for mouse CCR2 and CCR5, respectively.

### Assays for Inhibition of CCR2- and CCR5-Mediated Calcium Flux in Cells

Binding of MCP-1 to CCR2, or binding of MIP-1beta to CCR5, leads to a cascade of G protein-coupled signal transduction pathways. One of these is mobilization of calcium which is important for downstream cellular function, such as upregulation and activation of the integrin (CD11b). Intracellular calcium mobilization can be measured in the Fluorometric Imaging Plate Reader (FLIPR) as an increase in fluorescence emitted by the calcium-binding fluorophore (e.g. fluo-3) when cells preloaded with fluorophore are stimulated with MCP-1.

Human CCR2-mediated intracellular calcium flux assay was established with the human monocytic cell line, THP-1. THP-1 cells were first loaded with fluorophore by resuspending them in a glucose- and HEPES-buffered PBS (pH 7.4) containing 4 μM fluo-3 (Molecular Probes) and 1.25 mM probenecid and then incubated for 60 minutes at 37° C. After washing once to remove excess fluo-3, the cells were re-suspended in washing buffer (containing phenol red-free RPMI) with 1.25 mM probenecid, and plated into 96-well plate at 2×105/well. Compound C dilutions with a range of concentration from 0 to 100 nM or buffer alone were added to each well, centrifuged and incubated for 10 minutes. The plate was placed in a FLIPR-1™ (Molecular Devices) that uses an argon-ion laser to excite the cells and robotically adds human MCP-1 while monitoring changes in fluorescence. Recombinant human MCP-1 (PeproTech Inc.) was then added to a final concentration of 10 nM. The fluorescence shift was monitored and the base-to-peak excursion computed automatically. All samples were tested in duplicate. The inhibition achieved by graded concentrations of compound was calculated as a percentage of the compound-free MCP-1 control. A similar procedure to the above was adopted except that MIP-1beta (50 nM) was the ligand and the cell line was HT1080/CCR5 in which endogenous CCR5 is upregulated by random activation of gene expression (RAGE) technology.

Assays for inhibition of mouse CCR2- and to CCR5-mediated calcium flux in response to their respective ligand were established in similar fashion to the human assays except that WEHI-274.1 mouse monocyte line and L1.2 cells stably expressing mouse CCR5 were used as sources of mouse CCR2- and mouse CCR5-expressing cells, respectively, and mouse MCP-1 and -MIP-1beta were used as the ligands for mouse CCR2 and CCR5, respectively.

### Assays for Inhibition of CCR2- and CCR5-Mediated CD11b Integrin Upregulation in Whole Blood

A CCR2-dependent CD11b upregulation assay was established with human whole blood. Whole blood (100 μl) was pre-incubated with a concentration range of Compound C at 37° C. for 10 minutes. Human recombinant MCP-1 (10 μl of 100 nM) was then added to each reaction to a final concentration of 10 nM, except for unstimulated control reactions. The reactions were incubated for 30 minutes at 37° C. After incubation, 1 ml of ice cold FACS (PBS with 10% FBS) buffer was added, and the samples were centrifuged at 1500 rpm for 5 minutes and re-suspended in 50 μl of FACS buffer. The cells were then incubated with 20 μl of anti-CD14-FITC/anti-CD11 b-PE solution for 20 minutes on ice in the dark followed by addition of 1 ml of 1×FACS lysing solution (Becton Dickinson) to each reaction. The samples were then incubated for 30 minutes on ice in the dark. Following fixation and red blood cell lysis, the cells were centrifuged and re-suspended in 200 μl FACS-lysing solution. Samples were analyzed by flow cytometry within 1 hour of staining using a FACSCalibur™ flow cytometer. Data acquisition and analysis were performed using CellQuestPro™ software. A sequential gating strategy was used to analyze the CD14high CD11b+ monocyte population. For analysis, CD11b was measured as median fluorescence intensity (MFI). A similar procedure for mouse CCR5 was adopted except that MIP-1beta (50 nM) was used as the ligand.

A mouse CCR2-dependent CD11b upregulation assay was established with C57BL/6 mouse whole blood. Whole blood (100 ul) was pre-incubated with a concentration range of Compound C at 37° C. for 30 minutes. Mouse recombinant MCP-1 was then added to each reaction to a final concentration of 10 nM, except for unstimulated control reactions. The reactions were incubated for 15 minutes at 37° C. After incubation, 1 ml of ice cold FACS (PBS with 10% FBS) buffer was added, and the samples were centrifuged at 1500 rpm for 5 minutes and resuspended in 50 μl of FACS buffer. The cells were then incubated with 20 μl of anti-F4/80-PE/anti-mouse CD11b-APC solution for 20 minutes on ice in the dark followed by addition of 1 ml of 1×FACS lysing solution (Becton Dickinson) to each reaction. The samples were then incubated for 20 minutes on ice in the dark. Following fixation and red blood cell lysis, the cells were centrifuged and re-suspended in 200 ul FACS-lysing solution. Samples were analyzed within 1 hour of staining using a FACSCalibur™ flow cytometer. Data acquisition and analysis were performed using Flowjo software. A sequential gating strategy was used to analyze the F4/80+ CD11b+ monocyte population. For analysis, CD11b was measured as median fluorescence intensity (MFI). A similar procedure for mouse CCR5 was adopted as described for mouse CCR2 except the ligand was mouse MIP-1beta (50 nM).

Since Compound C has poor mouse PK and mouse CCR2 potency, Compound A was used as a mouse surrogate of CCR2/5-dual antagonist to evaluate as a monotherapy and in combination with anti-PD1 antibody in mouse models of tumor.

Some embodiments are directed to methods of treating cancer in a subject comprising administering to the subject a combination of a monoclonal antibody, a CCR2/5 dual antagonist, and/or chemotherapy.

Some embodiments are directed to methods of treating cancer in a subject comprising administering to the subject a combination of a monoclonal antibody and a CCR2/5 dual antagonist.

Some embodiments are directed to a combination of a monoclonal antibody, a CCR2/5 dual antagonist, and/or chemotherapy for use in treating cancer.

Some embodiments are directed to a combination of a monoclonal antibody and a CCR2/5 dual antagonist for use in treating cancer.

In some embodiments, the combination described herein can include administering more than one monoclonal antibody.

In some embodiments, the CCR2/5 dual antagonist is a compound of Formula (I), or a pharmaceutically acceptable salt thereof, or a combination thereof, administered to the subject in a single daily dose, or the total daily dosage may be administered in divided doses of two, three, or four times daily. In certain embodiments, the total daily dosage is administered one daily dose or twice a day. The amount of the compound of Formula (I), or pharmaceutically acceptable salt thereof, may be from about 100 mg per day to about 1200 mg per day. For example, the amount of the compound of Formula (I) administered to the subject may be about 10, 25, 50, 100, 200, 300, 400, 500, 600, 700, 800, 900, 1000, 1100, and 1200 mg per day. In certain embodiments, the amount of the compound of Formula (I) administered to the subject may be from about 10 mg to about 1200 mg per day; from about 25 mg to about 1200 mg per day; from about 50 mg to about 1200 mg per day; from about 100 mg to about 1200 mg per day; from about 200 mg to about 1200 mg per day; from about 300 mg to about 1200 mg per day; from about 300 mg to about 1200 mg per day; from about 400 mg to about 1200 mg per day; from about 500 mg to about 1200 mg per day; from about 600 mg to about 1200 mg per day. In certain embodiments, the amount of the compound of Formula (I) administered to the subject may be from about 10 mg to about 600 mg per day; from about 25 mg to about 600 mg per day; from about 50 mg to about 600 mg per day; from about 100 mg to about 600 mg per day; from about 200 mg to about 600 mg per day; from about 300 mg to about 600 mg per day. In certain embodiments, the amount of the compound of Formula (I) administered to the subject may be from about 10 mg to about 300 mg per day; from about 25 mg to about 300 mg per day; from about 50 mg to about 300 mg per day; from about 100 mg to about 300 mg per day; from about 200 mg to about 400 mg per day. In certain embodiments, the amount of the compound of Formula (I) is administered in doses of 300, and 600 mg either once or twice a day.

The amounts of the compound of Formula (I) described herein are based on the free form of the compound of Formula (I), that is, the non-salt form. If salts are administered, the amounts need to be calculated as a function of the molecular weight ratio between the salt and the free form.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia, for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, Jr., L. V., ed., Remington: The Science and Practice of Pharmacy, 22nd Edition, Pharmaceutical Press, London, UK (2012).

“Pharmaceutically acceptable excipient” refers to a diluent, adjuvant, excipient or carrier with which a compound of the disclosure is administered. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, stearates, silicon dioxide, polyvinyl alcohols, lubricant, talc, titanium dioxide, ferric oxide, and polyethylene glycols.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The term “antibody,” and like terms is meant in a broad sense and includes immunoglobulin molecules including, monoclonal antibodies (such as murine, human, human-adapted, humanized, and chimeric monoclonal antibodies), antibody fragments, bispecific or multispecific antibodies, dimeric, tetrameric or multimeric antibodies, and single chain antibodies. Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Monoclonal antibody” refers to a population of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

### Anti-PD-1 Antibodies Useful for the Invention

Any anti-PD-1 antibody that is known in the art may be used in the presently described methods. In particular, various human monoclonal antibodies that bind specifically to PD-1 with high affinity have been disclosed in U.S. Pat. No. 8,008,449. Each of the anti-PD-1 humanized antibodies disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-7 production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and (j) inhibits tumor cell growth in vivo. Anti-PD-1 antibodies usable in the present invention include monoclonal antibodies that bind specifically to human PD-1 and exhibit at least one, in some embodiments, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061.

In some embodiments, the anti-PD-1 antibody is selected from nivolumab (also known as “OPDIVO”; formerly designated 5C4, BMS-936558, MDX-1106, or ONO-4538), pembrolizumab (Merck, also known as “KEYTRUDA”, lambrolizumab, and MK-3475. See WO 2008/156712), PDR001 (Novartis; see WO 2015/112900), MEDI-0680 (AstraZeneca; AMP-514; see WO 2012/145493), cemiplimab (REGN-2810) (Regeneron; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (SHR-1210; Jiangsu Hengrui Medicine; see WO 2015/085847; Si-Yang Liu et al., J Hematol. Oncol. 10:136 (2017)), TSR-042 (ANBO11; Tesaro Biopharmaceutical; see WO2014/179664), GLS-010 (WBP3055; Wuxi/Harbin Gloria Pharmaceuticals; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGD013 (Macrogenics) and IB1308 (Innovent; see WO 2017/024465, WO 2017/025016, WO 2017/132825, WO2017/133540).

In some embodiments, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In other embodiments, the anti-PD-1 antibody is pembrolizumab. Pembrolizumab is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also www.cancer.gov/drugdictionary?cdrid=695789 (last accessed: Dec. 14, 2014). Pembrolizumab has been approved by the FDA for the treatment of relapsed or refractory melanoma.

Anti-PD-1 antibodies usable in the disclosed methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the methods of the disclosed invention also include antigen-binding portions of the above antibodies. It has been amply demonstrated that the antigen-binding function of an antibody can be performed by fragments of a full-length antibody.

Anti-PD-1 antibodies suitable for use in the disclosed methods or compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain embodiments, the anti-PD-1 antibody or antigen-binding portion thereof cross-competes with nivolumab for binding to human PD-1.

### Antibody B

Due to lack of cross-reactivity of nivolumab to mouse PD-1, an anti-mouse PD-1 antibody (hereinafter referred to as Antibody B) was generated by murinizing the Fc tail of 4H2, a rat-anti-mouse PD1 antibody. Antibody B contains a mouse IgG1 D265A, and comprises variable region light and heavy chain sequences as shown below.

Antibody B light chain variable region:
(SEQ ID NO: 1)
DTVLTQSPALAVSLGQRVTISCKASETVSSSMYSYIHWYQQKPGQQPKLL
IYRASNLESGVPARFSGSGSGTDFTLTIDPVEADDVATYFCQQSWNPWTF
GGGTKLELKRADAAPTVSIFPPSSEQLTSGGASVVCFLNNFYPKDINVKW
KIDGSERQNGVLNSWTDQDSKDSTYSMSSTLTLTKDEYERHNSYTCEATH
KTSTSPIVKSFNRNEC
Antibody B heavy chain variable region:
(SEQ ID NO: 2)
QVQLKESGPGLVQPSQTLSLTCTVSGFSLTSYNVHWVRQPPGKGLEWMGG
MRYNEDTSYNSALKSRLSISRDTSKNQVFLKMNSLQTDDTGTYYCTRDAV
YGGYGGWFAYWGQGTLVTVSSAKTTPPSVYPLAPGSAAQTNSMVTLGCLV
KGYFPEPVTVTWNSGSLSSGVHTFPAVLQSDLYTLSSSVTVPSSTWPSET
VTCNVAHPASSTKVDKKIVPRDCGCKPCICTVPEVSSVFIFPPKPKDVLT
ITLTPKVTCVVVDISKDDPEVQFSWFVDDVEVHTAQTQPREEQFNSTFRS
VSELPIMHQDWLNGKEFKCRVNSAAFPAPIEKTISKTKGRPKAPQVYTIP
PPKEQMAKDKVSLTCMITDFFPEDITVEWQWNGQPAENYKNTQPIMDTDG
SYFVYSKLNVQKSNWEAGNTFTCSVLHEGLHNHHTEKSLSHSPGK

Antibody B was used as an anti-PD1 mouse surrogate to study the efficacy of CCR2/5-dual antagonist in combination with anti-PD1 antibody. 4H2 exhibited an EC50 of 2.9 nM in binding to mouse PD1-expressing CHO cells, which is comparable to that of nivolumab binding to human PD1-expressing CHO cells (0.4 nM). In addition, 4H2 exhibited an IC50 of 3.6 and 4.9 nM in blocking mouse PD1 binding to mouse PD-L1 and mouse PD-L2, respectively, which comparable to nivolumab binding to human PD-L1 and human PD-L2 (1.04 and 0.97 nM, respectively) (Table 2)

TABLE 2
Mouse potency of antibody B versus human potency of Nivolumab

| | Antibody | Nivolumab |
|---|---|---|
| | B mouse | human |
| Assay | potency | potency |
| Direct binding to PD1- | 2.9 nM | 0.4 nM |
| expressing cells for EC50 | | |
| Blocking PD-Ll binding to | 3.6 nM | 1.04 nM |
| PD1-expressing cells for IC50 | | |
| Blocking PD-L2 binding to | 4.9 nM | 0.97 nM |
| PD1-expressing cells for IC50 | | |

Assays for Direct Binding of Anti-PD-1 Monoclonal Antibody (mAb) to PD1 Stably Expressed on CHO Transfectants

mAbs against human and mouse PD-1 were serially diluted and incubated with CHO cell transfectants stably expressing human and mouse PD-1, respectively, followed by detection with a FITC-conjugated secondary to mouse IgG Fcγ.

### Assays for Blocking Binding of PD-L1 to PD-1 Stably Expressed on CHO Transfectants

CHO transfectants stably expressing human and mouse PD-1 were pre-incubated with titrated mAbs against human and mouse PD-1, respectively, followed by addition of PD-L1-Fc at 2 μg/mL. Cell-bound PD-L1-Fc was detected with a FITC-conjugated secondary to human IgG Fcγ.

### Assays for Blocking Binding of PD-L2 to PD-1 Stably Expressed on CHO Transfectants

CHO transfectants stably expressing human and mouse PD-1 were pre-incubated with titrated mAbs against human and mouse PD-1, respectively, followed by addition of PD-L2-Fc at 15 μg/ml. Cell-bound PD-L2-Fc was detected with a FITC-conjugated secondary to human IgG Fc.

### Anti-PD-L1 Antibodies Useful for the Invention

Any anti-PD-L1 antibody may be used in the methods of the present disclosure. Examples of anti-PD-L1 antibodies useful in the methods of the present disclosure include the antibodies disclosed in U.S. Pat. No. 9,580,507. Each of the anti-PD-L1 human monoclonal antibodies disclosed in U.S. Pat. No. 9,580,507 have been demonstrated to exhibit one or more of the following characteristics: (a) binds to human PD-L1 with a K_{D} of 1×10⁻⁷ M or less, as determined by surface plasmon resonance using a Biacore biosensor system; (b) increases T-cell proliferation in a Mixed Lymphocyte Reaction (MLR) assay; (c) increases interferon-7 production in an MLR assay; (d) increases IL-2 secretion in an MLR assay; (e) stimulates antibody responses; and (f) reverses the effect of T regulatory cells on T cell effector cells and/or dendritic cells. Anti-PD-L1 antibodies usable in the present invention include monoclonal antibodies that bind specifically to human PD-L1 and exhibit at least one, in some embodiments, at least five, of the preceding characteristics.

In certain embodiments, the anti-PD-L1 antibody is selected from the group consisting of BMS-936559 (formerly 12A4 or MDX-1105; see, e.g., U.S. Pat. No. 7,943,743 and WO 2013/173223), MPDL3280A (also known as RG7446, atezolizumab, and TECENTRIQ; U.S. Pat. No. 8,217,149; see, also, Herbst et al. (2013) J Clin Oncol 31(suppl):3000), durvalumab (IMFINZI; MEDI-4736; AstraZeneca; see WO 2011/066389), avelumab (Pfizer; MSB-0010718C; BAVENCIO; see WO 2013/079174), STI-1014 (Sorrento; see WO2013/181634), CX-072 (Cytomx; see WO2016/149201), KN035 (3D Med/Alphamab; see Zhang et al., CellDiscov. 7:3 (March 2017), LY3300054 (Eli Lilly Co.; see, e.g, WO 2017/034916), and CK-301 (Checkpoint Therapeutics; see Gorelik et al., AACR:Abstract 4606 (April 2016)).

In certain embodiments, the PD-L1 antibody is atezolizumab (TECENTRIQ). Atezolizumab is a fully humanized IgG1 monoclonal anti-PD-L1 antibody.

In certain embodiments, the PD-L1 antibody is durvalumab (IMFINZI). Durvalumab is a human IgG1 kappa monoclonal anti-PD-L1 antibody.

In certain embodiments, the PD-L1 antibody is avelumab (BAVENCIO). Avelumab is a human IgG1 lambda monoclonal anti-PD-L1 antibody.

In other embodiments, the anti-PD-L1 monoclonal antibody is selected from the group consisting of 28-8, 28-1, 28-12, 29-8, 5H1, and any combination thereof.

Anti-PD-L1 antibodies usable in the disclosed methods also include isolated antibodies that bind specifically to human PD-L1 and cross-compete for binding to human PD-L1 with any anti-PD-L1 antibody disclosed herein, e.g., atezolizumab and/or avelumab. In some embodiments, the anti-PD-L1 antibody binds the same epitope as any of the anti-PD-L1 antibodies described herein, e.g., atezolizumab and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1 with, or bind to the same epitope region of human PD-L1 antibody as, atezolizumab and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the methods of the disclosed invention also include antigen-binding portions of the above antibodies. It has been amply demonstrated that the antigen-binding function of an antibody can be performed by fragments of a full-length antibody.

Anti-PD-L1 antibodies suitable for use in the disclosed methods or compositions are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab and/or avelumab for binding to human PD-L1.

### Anti-CTLA-4 Antibodies Useful for the Invention

Any anti-CTLA-4 antibody that is known in the art may be used in the methods of the present disclosure. Anti-CTLA-4 antibodies of the instant invention bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

In certain embodiments, the CTLA-4 antibody is selected from ipilimumab (YERVOY; U.S. Pat. No. 6,984,720), MK-1308 (Merck), AGEN-1884 (Agenus Inc.; WO 2016/196237), and tremelimumab (formerly ticilimumab, CP-675,206; AstraZeneca; see, e.g., WO 2000/037504 and Ribas, Update Cancer Ther. 2(3): 133-39 (2007)). In particular embodiments, the anti-CTLA-4 antibody is ipilimumab.

In particular embodiments, the CTLA-4 antibody is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

In particular embodiments, the CTLA-4 antibody is MK-1308, which is an anti-CTLA-4 antibody under development by Merck.

In particular embodiments, the CTLA-4 antibody is AGEN-1884, which is a recombinant human monoclonal antibody to human CTLA-4, developed by Agenus Inc.

Anti-CTLA-4 antibodies usable in the disclosed methods also include isolated antibodies that bind specifically to human CTLA-4 and cross-compete for binding to human CTLA-4 with any anti-CTLA-4 antibody disclosed herein, e.g., ipilimumab and/or tremelimumab. In some embodiments, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain embodiments, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the methods of the disclosed invention also include antigen-binding portions of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain embodiments, the anti-CTLA-4 antibody or antigen-binding portion thereof cross-competes with ipilimumab and/or tremelimumab for binding to human CTLA-4.

As used herein, “combination” is meant to include therapies that can be administered separately, for example, formulated separately for separate administration (e.g., as may be provided in a kit), and therapies that can be administered together in a single formulation (i.e., a “co-formulation”). In certain aspects, the monoclonal antibody and the compound of Formula (I), or pharmaceutically acceptable salt thereof, and/or chemotherapy, are administered or applied sequentially, e.g., where one agent is administered prior to one or more other agents. In other embodiments, the monoclonal antibody and the compound of Formula (I), or pharmaceutically acceptable salt thereof, and/or chemotherapy, are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

In exemplary aspects of the disclosure, the antibody is nivolumab. In some aspects, the nivolumab may be administered by intravenous infusion at a dose of about 400 mg to 500 mg every 4 weeks. For example, the nivolumab may be administered to the subject by intravenous infusion at a dose of about 400 mg, 410 mg, 420 mg, 430 mg, 440 mg, 450 mg, 460 mg, 470 mg, 480 mg, 490 mg, or about 500 mg every 4 weeks. In preferred aspects, the nivolumab may be administered by intravenous infusion at a dose of about 480 mg every 4 weeks.

In some aspects, the nivolumab may be administered to the subject by intravenous infusion at a dose of about 80 mg to 360 mg every 3 weeks. For example, the nivolumab may be administered to the subject by intravenous infusion at a dose of about 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, or about 360 mg every 3 weeks. In preferred aspects, nivolumab may be administered by intravenous infusion at a dose of about 80 mg every 3 weeks. In other preferred aspects, the nivolumab is administered by intravenous infusion at a dose of about 360 mg every 3 weeks.

In some aspects, the nivolumab may be administered by intravenous infusion at a dose of about 200 mg to 300 mg every 2 weeks. For example, the nivolumab may be administered to the subject by intravenous infusion at a dose of about 200 mg, 210 mg, 220 mg, 230 mg, 240 mg, 250 mg, 260 mg, 270 mg, 280 mg, 290 mg, or about 300 mg every 2 weeks. In preferred aspects, the nivolumab may be administered by intravenous infusion at a dose of about 240 mg every 2 weeks.

In some aspects, the nivolumab is further administered with ipilimumab, wherein the ipilimumab may be administered by intravenous infusion at a dose of about 1 mg/kg to 10 mg/kg every 3 weeks. For example, the ipilimumab may be administered to the subject by intravenous infusion at a dose of about 1 mg/kg, 2 mg/kg, 3 mg/kg, 4 mg/kg, 5 mg/kg, 6 mg/kg, 7 mg/kg, 8 mg/kg, 9 mg/kg or 10 mg/kg every 3 weeks. In preferred aspects, the ipilimumab may be administered by intravenous infusion at a dose of about 3 mg/kg every 3 weeks.

As used herein, “administered to the subject” and similar terms indicate a procedure by which the compound is injected into a patient such that target cells, tissues, or segments of the body of the subject are contacted with the compound. Methods of administration contemplated herein include, but are not limited to, oral, local, inhalation, or parenteral administration. Suitable parenteral methods of administration include, but are not limited to, intravenous, intramuscular, subcutaneous and intradermal parental administration.

In some aspects, a combination of a monoclonal antibody and a compound of Formula (I), or a pharmaceutically acceptable salt thereof, as described herein may be administered to a subject with cancer, wherein the subject may have been previously administered at least one prior therapy for the treatment of cancer. Such subjects may be referred to as “treatment experienced” or “non-treatment-naïve.” In some aspects, the prior therapy is ongoing. In other aspects, the prior therapy has been discontinued. In these subjects, the prior therapy may have been discontinued for about 12 or 24 hours. In other aspects, the prior therapy may have been discontinued for about 2, 3, 4, 5, or 6 days. In other aspects, the prior therapy may have been discontinued for about 1, 2, 3, 4, 5, 6, 7, or 8 weeks or longer. In some aspects, the prior therapy may have been discontinued for about 3, 4, 5, 6, 7, 8, 9, 10, or about 11 months. In other aspects, the prior therapy may have been discontinued for about 1, 2, 3, 4, 5, 6, 7, 8, 9, or about 10 years.

Examples of the prior therapies include, but are not limited to: surgery, radiotherapy, chemotherapy, immunotherapy, targeted therapy, hormone therapy, stem cell transplant or precision medicine treatment.

As used herein, “chemotherapy” refers to the administration of one or more chemotherapeutic drugs and/or other agents to a cancer patient by various methods, including intravenous, oral, intramuscular, intraperitoneal, intravesical, subcutaneous, transdermal, buccal, inhalation, or in the form of a suppository.

As used herein, “surgery” refers to surgical methods employed to remove cancerous tissue, including but not limited to tumor biopsy or removal of part or all of the colon (colostomy), bladder (cystectomy), spleen (splenectomy), gallbladder (cholecystectomy), stomach (gastrectomy), liver (partial hepatectomy), pancreas (pancreatectomy), ovaries and fallopian tubes (bilateral salpingooophoroectomy), omentum (omentectomy) and/or uterus (hysterectomy).

In some aspects, a combination of a monoclonal antibody and a compound of Formula (I), or a pharmaceutically acceptable salt thereof, as described herein may be administered to a subject with cancer, wherein the subject is treatment naïve. As used herein, “treatment naïve,” means that the subject was not previously administered a prior therapy for the treatment of the cancer.

In certain aspects, the monoclonal antibody and a compound of Formula (I), or a pharmaceutically acceptable salt thereof, may be administered in further combination with additional therapeutic agents in any manner appropriate under the circumstances. Examples of therapeutic agents that may be used in combinations for treating cancers disclosed herein include radiation, an immunomodulatory agent or chemotherapeutic agent, or diagnostic agent. Suitable immunomodulatory agents that may be used in the present invention include other monoclonal antibodies described herein, CD40L, B7, and B7RP1; activating monoclonal antibodies (mAbs) to stimulatory receptors, such as, ant-CD40, anti-CD38, anti-ICOS, and 4-IBB ligand; dendritic cell antigen loading (in vitro or in vivo); anti-cancer vaccines such as dendritic cell cancer vaccines; cytokines/chemokines, such as, IL1, IL2, IL12, IL18, ELC/CCL19, SLC/CCL21, MCP-1, IL-4, IL-18, TNF, IL-15, MDC, IFNa/b, M-CSF, IL-3, GM-CSF, IL-13, and anti-IL-10; bacterial lipopolysaccharides (LPS); and immune-stimulatory oligonucleotides.

Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine and floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT 11; topoisomerase inhibitors; difluoromethylornithine (DMFO); retinoic acid; esperamicins; capecitabine; and pharmaceutically acceptable salts, acids or derivatives of any of the above.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above. In certain embodiments, combination therapy comprises administration of a hormone or related hormonal agent.

Chemotherapeutic agents also include signal transduction inhibitors (STI). The term “signal transduction inhibitor” refers to an agent that selectively inhibits one or more steps in a signaling pathway. Signal transduction inhibitors (STIs) of the present invention include: (i) bcr/abl kinase inhibitors (e.g., GLEEVEC™); (ii) epidermal growth factor (EGF) receptor inhibitors, including kinase inhibitors and antibodies; (iii) her-2/neu receptor inhibitors (e.g., HERCEPTIN™); (iv) inhibitors of Akt family kinases or the Akt pathway (e.g., rapamycin); (v) cell cycle kinase inhibitors (e.g., flavopiridol); and (vi) phosphatidyl inositol kinase inhibitors.

Chemotherapeutic agents also include oxaliplatin, a vitamin B derivative, e.g., leucovorin (FOL, folinic acid), and a topoisomerase inhibitor, e.g., irinotecan.

In some embodiments, the chemotherapeutic agents are selected from paclitaxel, gemcitabine, 5-fluorouracil (5-FU), leucovorin, and irinotecan.

FOLFIRI is a chemotherapy regimen comprising FOL—folinic acid (leucovorin) and F—fluorouracil (5-FU), and IRI—irinotecan.

FOLFOX is a chemotherapy regimen comprising FOL—folinic acid (leucovorin) and F—fluorouracil (5-FU), and OX—oxaliplatin.

Gem/ABRAXANE (nab-paclitaxel) is a chemotherapy regimen comprising gemcitabine and nab-paclitaxel.

Additional treatment modalities that may be used in combination with a monoclonal antibody and a compound of Formula (I), or a pharmaceutically acceptable salt thereof, include a cytokine or cytokine antagonist, such as IL-12, IFN, or anti-epidermal growth factor receptor, radiotherapy, a monoclonal antibody against another tumor antigen, a complex of a monoclonal antibody and toxin, a T-cell adjuvant, bone marrow transplant, or antigen presenting cells (e.g., dendritic cell therapy). Vaccines (e.g., as a soluble protein or as a nucleic acid encoding the protein) are also provided herein.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” pancreatic cancer refers to a tumor that is the result of a metastasized pancreatic cancer Because the distal tumor is derived from the pre-metastasis tumor, the “derived from” tumor can also comprise the pre-metastasis tumor, e.g., a tumor derived from a pancreatic cancer can comprise a pancreatic cancer.

In some aspects, the cancer is a malignant solid tumor. In further aspects, the cancer is metastatic and/or unresectable. Examples of the cancers that may be treated using the compounds and compositions described herein include, but are not limited to: pancreatic cancer, colorectal cancer, non-small cell lung cancer, renal cell carcinoma; squamous cell carcinoma of the head and neck, bladder cancer, cancers of the prostate, cervix, stomach, endometrium, brain, liver, ovary, testis, head, neck, skin (including melanoma and basal carcinoma), mesothelial lining, esophagus, breast, muscle, connective tissue, lung (including small-cell lung carcinoma and non-small-cell carcinoma), adrenal gland, thyroid, kidney, or bone; glioblastoma, mesothelioma, gastric cancer, sarcoma, choriocarcinoma, cutaneous basocellular carcinoma, and testicular seminoma. In preferred aspects, the cancer is cervical cancer, non-small cell lung cancer, renal cell carcinoma; squamous cell carcinoma of the head and neck, bladder cancer, pancreatic cancer, melanoma, lymphoma or gastric cancer. In more preferred aspects, the cancer is melanoma, non-small cell lung cancer, squamous cell carcinoma of the head and neck, bladder cancer, renal cell carcinoma or gastric carcinoma.

In some aspects of the disclosure, the subject exhibits an improvement in his/her Eastern Cooperative Oncology Group (ECOG) Performance Status following treatment according to any of the disclosed methods. In some aspects, the subject exhibits an ECOG Performance Status of less than or equal to 1 following treatment as described herein. In other aspects, subject exhibits an ECOG Performance Status of less than or equal to 2 following treatment. In other aspects, subject exhibits an ECOG Performance Status of less than or equal to 3 following treatment. In other aspects, subject exhibits an ECOG Performance Status of less than or equal to 4 following treatment. ECOG Performance Status, developed by the Eastern Cooperative Oncology Group, provides the following status descriptions per grade: Grade 0 is fully active, able to carry on all pre-disease performance without restriction; Grade 1 is restricted in physically strenuous activity but ambulatory and able to carry out work of a light or sedentary nature, e.g., light house work, office work; Grade 3 is ambulatory and capable of all self-care but unable to carry out any work activities; up and about more than 50% of waking hours.

In some aspects, the subject is an adult. For example, adult populations may include subjects aged 18 and older. In other aspects, the subject is a geriatric subject. For example, geriatric populations may include subjects aged 64 and older. In other aspects, the subject is a pediatric subject. For example, pediatric subjects may be preterm neonatal (the period at birth when a newborn is born before the full gestational period), term neonatal (birth to 27 days), an infant (28 days to 12 months), a toddler (13 months to 2 years), in early childhood (2 years to 5 years), in middle childhood (6 years to 11 years), in early adolescence (12 years to 18 years), or in late adolescence (19 years to 21 years).

In some aspects, the methods of treatment disclosed herein may result in a treatment related adverse event (TRAE) as established by the Common Terminology Criteria for Adverse Events (CTCAE), published by the U.S. Department of Health and Human Services. An Adverse Events (AE) is any unfavorable and unintended sign (including an abnormal laboratory finding), symptom, or disease temporally associated with the use of a medical treatment or procedure that may or may not be considered related to the medical treatment or procedure. An AE is a term that is a unique representation of a specific event used for medical documentation and scientific analyses. The general guidelines as established by the CTCAE are as follows: Grade refers to the severity of the AE where grade 1 is defined as mild; asymptomatic or mild symptoms; clinical or diagnostic observations only; intervention not indicated. Grade 2 is defined as moderate; minimal, local or noninvasive intervention indicated; limiting age-appropriate instrumental activities of daily living (ADL). Grade 3 is severe or medically significant but not immediately life threatening; hospitalization or prolongation of hospitalization indicated; disabling; limiting self care ADL. Grade 4 is life-threatening consequences; urgent intervention indicated. Grade 5 is death related to AE. Examples of TRAE's greater than or equal to 2 include uveitis, decreased appetite, pyrexia, anemia, autoimmune hepatitis, fatigue, headache, nausea and/or vomiting. Examples of Grade 3/4 TRAEs, also referred to as “serious TRAEs” include increase lipase, hypophosphatemia, rash, increased aspartate aminotransferase, increased alanine aminotransferase, hepatitis, hypertension, pancreatitis, and/or autoimmune hepatitis.

In certain aspects, the treatments described herein may not result in a grade 4 or grade 5 adverse event. In other aspects, the treatments described herein may result in no more than a grade 1 adverse event. In further aspects, the treatments described herein may result in no more than a grade 2 adverse event. In still further aspects, the treatments described herein may result in no more than a grade 3 adverse event.

In aspects of the methods disclosed herein, the subject may exhibit improved anti-tumor activity as measured by objective response rate (ORR), duration of response, and progression-free survival (PFS) rate.

The objective response rate (ORR) may be quantified by an investigator and/or physician to assess response using Response Evaluation Criteria In Solid Tumors (RECIST) v1.1, as developed by a collaboration between the European Organization for Research and Treatment of Cancer (EORTC), the National Cancer Institute (NCI), and the United States and the National Cancer Institute of Canada Clinical Trials Group. Optionally, the ORR may optionally be reviewed by a central imaging lab.

“Progression free survival (PFS),” as used in the context of the cancers described herein, refers to the length of time during and after treatment of the cancer until objective tumor progression or death. The treatment may be assessed by objective or subjective parameters; including the results of a physical examination, neurological examination, or psychiatric evaluation. In preferred aspects, PFS may be assessed by blinded imaging central review and may further optionally be confirmed by ORR or by blinded independent central review (BICR).

“Overall survival (OS)” may be assessed by OS rate at certain time points (e.g., 1 year and 2 years) by the Kaplan-Meier method and corresponding 95% CI will be derived based on Greenwood formula by study treatment for each tumor type. OS rate is defined as the proportion of participants who are alive at the time point. OS for a participant is defined as the time from the first dosing date to the date of death due to any cause.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

“Subtherapeutic dose” means a dose of a therapeutic compound (e.g., an antibody) that is lower than the usual or typical dose of the therapeutic compound when administered alone for the treatment of a hyperproliferative disease (e.g., cancer).

In some embodiments, the methods disclosed herein are used in place of standard of care therapies. In certain embodiments, a standard of care therapy is used in combination with any method disclosed herein. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014, available at: www.nccn.org/professionals/physician_gls/f_guidelines.asp, last accessed May 14, 2014).

Treatment is continued as long as clinical benefit is observed or until unacceptable toxicity or disease progression occurs. In certain embodiments, the anti-PD-1 antibody can be administered at the dosage that has been shown to produce the highest efficacy as monotherapy in clinical trials, e.g., about 3 mg/kg of nivolumab administered once about every three weeks (Topalian et al., 2012 N Engl J Med 366:2443-54; Topalian et al., 2012 Curr Opin Immunol 24:207-12), at a flat dose of 240 mg, or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health, and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### Kits

Also within the scope of the present disclosure are kits comprising a CCR2/5 dual antagonist a monoclonal antibody and/or chemotherapeutic agents for therapeutic uses. Kits typically include a label indicating the intended use of the contents of the kit and instructions for use. The term label includes any writing, or recorded material supplied on or with the kit, or which otherwise accompanies the kit.

The following examples are merely illustrative and are not intended to limit the disclosure to the materials, conditions, or process parameters set forth therein.

### EXAMPLES

### Example 1. Combination of Compound A and Antibody B Against Mouse Colon Tumor (MC38) Progression

Combination pharmacology studies were conducted to evaluate a CCR2/5-dual antagonist, Compound A, in combination with an anti-PD-1 antibody, Antibody B, in tumor bearing mice.

Female C57BL/6 mice from Charles River Laboratories (Raleigh, N.C.) were received in house at age 6-8 weeks and acclimated for 3-7 days prior to implant. Mouse colon tumor MC38 cells were implanted subcutaneously at a concentration of 1×10⁷ cells/mL, 0.1 mL per injection, using a 1 mL tuberculin syringe with 25 g needle.

On Day 6 post implant, mice were randomized and sorted into groups with 10 mice per group. Treatment was initiated on Day 6 with control vehicle and Isotype control; Antibody B (anti-mouse PD1) 10 mg/kg alone; Compound A at 25, 50, and 100 mg/kg alone (for Study #1), at 6.25, 12.5 25 and 50 mg/kg (for Study #2); Compound A at 25, 50, and 100 mg/kg in combination with antibody B at 10 mL/kg (for Study #1), at 6.25, 12.5, 25 and 50 mg/kg in combination with Antibody B at 10 mg/kg (for Study #2). Compound A was administered on a continuous schedule twice daily by oral dosing for 28 days. Antibody B was administered i.p. every 4 days for a total of 3 doses. Blood was collected (10 μL tail bleed) onto DBS (dried blood spot) cards at the midpoint of the experiment for Compound A PK evaluation at time points of 1, 4, 7, and 24 hours.

Tumors and group body weights were weighed and measured twice weekly until tumors reached a volume of approximately 1500 mm³. Animals were euthanized if the tumor reached a volume greater than approximately 1500 mm³ or appeared ulcerated. Mean, median and/or survival plots as well as number of tumor-free mice were calculated to determine efficacy.

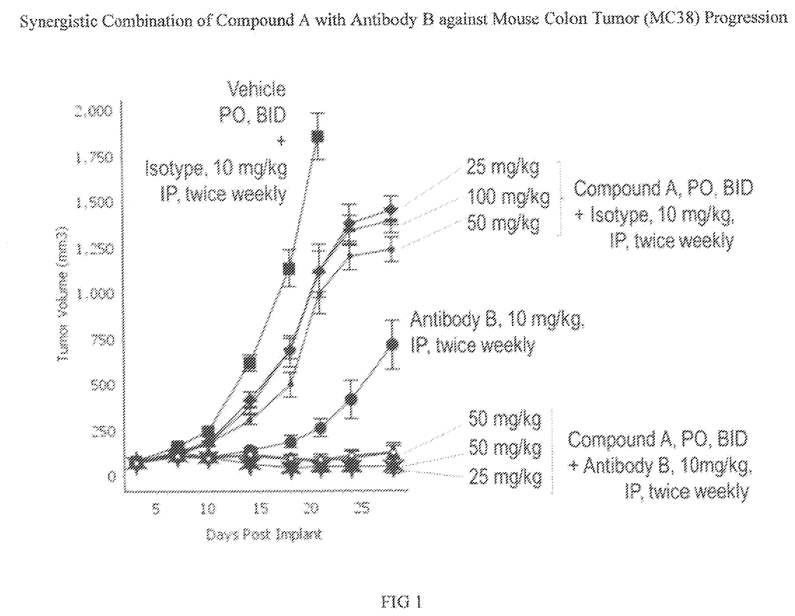

Results from these studies suggest that combination of Compound A, dosed PO BID at 25, 50 and 100 mg/kg for 28 days, respectively, and Antibody B, dosed twice weekly at 10 mg/kg for a total of 3 doses provide greater anti-tumor efficacy relative to either agent alone, as measured by reduction in tumor volume (see FIG. 1 Error! Reference source not found).

Analysis of trough exposure of Compound A showed 0.6-, 2.5- and 26.5-fold IC90 at doses 25, 50 and 100 mg/kg, respectively, indicating that Compound A with a trough coverage between 0.6- and 26.5-fold IC90 synergizes with Antibody B against tumor progression (Table 1). Both Compound A and Antibody B, alone or in combination, were well tolerated. None of the mice treated with the combination showed any clinical signs of toxicity and there were no effects on bodyweight.

TABLE 1
Doses, anti-tumor efficacy and exposures/trough coverage of
Compound A alone and in combination with Antibody B

| | Compound A | | | Antibody B | Compound A + Antibody B | | |
|---|---|---|---|---|---|---|---|
| Dose (mg/kg) | 25 | 50 | 100 | 10 | 25 | 50 | 100 |
| Tumor-free mice | 0/10 | 0/10 | 0/10 | 1/10 | 8/10 | 4/10 | 2/10 |
| IC90-fold @ trough | 0.4 | 4.2 | 25.4 | n/a | 0.6 | 2.5 | 26.5 |

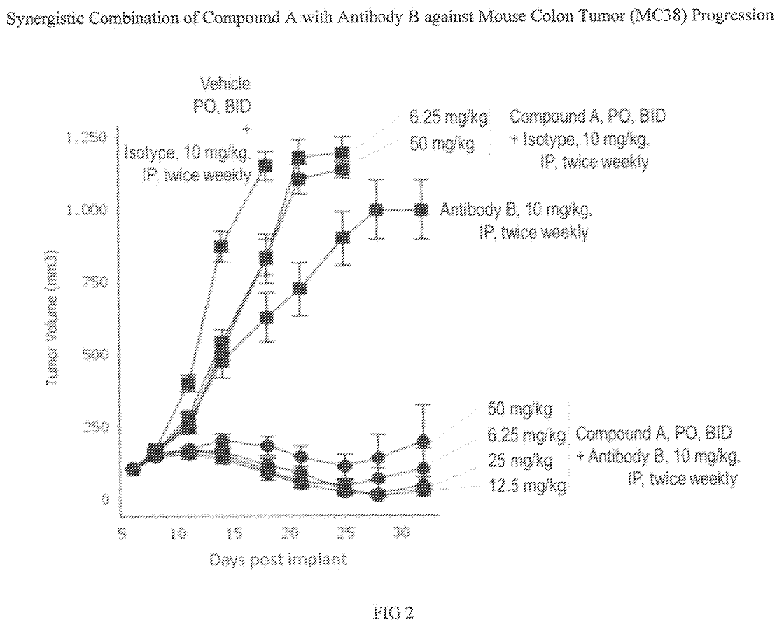

Another study with lower doses of Compound A in combination with Antibody B was conducted and the results showed that combination of Compound A dosed PO BID at 6.25, 12.5 25, and 50 mg/kg for 28 days, respectively, and Antibody B dosed twice weekly at 10 mg/kg for a total of 3 doses, provide greater anti-tumor efficacy relative to either agent alone, as measured by reduction in tumor volume (see FIG. 2).

Analysis of trough exposure of Compound A showed 0.6-, 2.5- and 26.5-fold IC90 at doses 6.25 12.5 25, and 50 mg/kg, respectively, indicating that Compound A with a trough coverage between 0.05- and 1.56-fold IC90 synergizes with Antibody B against tumor progression (Table 2).

TABLE 2
Doses, anti-tumor efficacy and exposures/trough coverage of
Compound A alone and in combination with Antibody B

| | Compound A | | Antibody B | Compound A + Antibody B | | | |
|---|---|---|---|---|---|---|---|
| Dose mg/kg | 6.25 | 50 | 10 | 6.25 | 12.5 | 25 | 50 |
| Tumor-free | 0/10 | 0/10 | 0/10 | 8/10 | 8/8 | 8/8 | 4/9 |
| mice | | | | | | | |
| IC90-fold @ trough | 0.1 | 5.4 | n.d. | 0.05 | 0.15 | 0.34 | 1.56 |
| IC50-fold @ trough | 0.9 | 48.6 | n.d. | 0.5 | 1.3 | 3.1 | 14.0 |

n.d.: not determined

### Example 2. Combination of Compound A and Antibody B against Mouse Colon Tumor (CT26) Progression

Combination pharmacology studies were conducted to evaluate Compound A in combination with Antibody B in CT26 colon tumor model.

Female BALB/C mice from ENVIGO (Frederick, Md.) were received in house at age 6-8 weeks and acclimated for 3-7 days prior to implantation. Mouse colon tumor CT26 cells were implanted subcutaneously at a concentration of 1×107 cells/mL, 0.1 mL per injection, using a 1 mL tuberculin syringe with 25 g needle.

On Day 10 post implant, mice were randomized and sorted into groups with 10 mice per group. Treatment was initiated on Day 10 with control vehicle and Isotype control; Antibody B (anti-mouse PD1) 10 mg/kg alone; Compound A at 6.25, 12.5, 25 and 50 mg/kg alone. Compound A at 6.25, 12.5, 25 and 50 mg/kg in combination with antibody B at 10 mL/kg. Compound A was administered on a continuous schedule twice daily by oral dosing for 28 days. Antibody B was administered i.p. every 4 days for a total of 3 doses.

Tumors and group body weights were weighed and measured twice weekly until tumors reached a volume of approximately 1500 mm³. Animals were euthanized if the tumor reached a volume greater than approximately 1500 mm³ or appeared ulcerated. Mean, median and/or survival plots as well as number of tumor-free mice were calculated to determine efficacy.

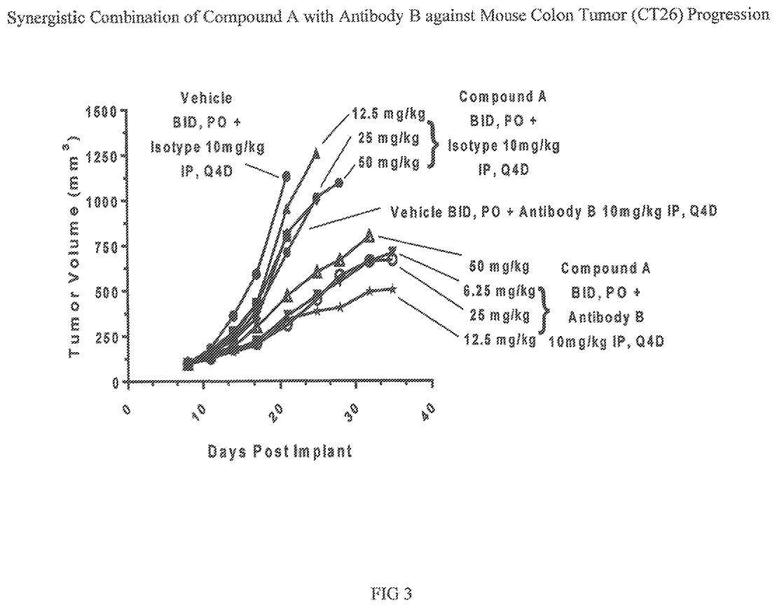

Results from these studies suggest that combination of Compound A, dosed PO BID 6.25, 12.5, 25 and 50 mg/kg for 28 days, respectively, and Antibody B, dosed twice weekly at 10 mg/kg for a total of 3 doses provide greater anti-tumor efficacy relative to either agent alone, as measured by reduction in tumor volume (see FIG. 3 and TABLE 3 Error! Reference source not found.), with the group of 12.5 mg/kg compound A combination with antibody B showing the most robust anti-tumor activity of the four combinations groups.

Based on PK findings with Compound A in BALB/C mice, its trough exposures are projected to give showed approximately 0.1-, 0.2-, 0.5-, and 1-fold IC90 at doses 6.25, 12.5, 25, and 50 mg/kg, respectively, indicating that Compound A with a trough coverage between 0.1- and 1-fold IC90 synergizes with Antibody B against tumor progression (Table 3). Both Compound A and Antibody B, alone or in combination, were well tolerated. None of the mice treated with the combination showed any clinical signs of toxicity and there were no effects on bodyweight.

TABLE 3
Doses, anti-tumor efficacy, trough coverage of
Compound A alone and in combination with Antibody B

| | Compound A | | | Antibody B | Compound A + Antibody B | | | |
|---|---|---|---|---|---|---|---|---|
| Dose mg/kg | 12.5 | 25 | 50 | 10 | 6.25 | 12.5 | 25 | 50 |
| Tumor-free | 0/10 | 0/10 | 0/10 | 0/10 | 1/10 | 3/10 | 0/10 | 0/10 |
| mice | | | | | | | | |
| IC90-fold @ trough | ~0.2 | ~0.5 | ~1 | n.d. | ~0.1 | ~0.2 | ~0.5 | ~1 |
| (Projected) | | | | | | | | |
| IC50-fold @ trough | ~2 | ~4 | ~8 | n.d. | ~1 | ~2 | ~4 | ~8 |
| (Projected) | | | | | | | | |

### Example 3. Compound C Administered in Combination with Either Nivolumab or Chemotherapy in Patients with Advanced Cancers

A non-limiting example of a phase 1b/2 open-label, 2-part, clinical trial, is described below.

### Purpose:

The purpose of this study is to, among other things, evaluate the safety profile, tolerability, PK, PD, and preliminary efficacy of Compound C, administered in combination with either nivolumab or chemotherapy in patients with metastatic colorectal and pancreatic cancers.

### Intervention:

Patients are administered Compound C at a specified dose at specified intervals. In some embodiments, the patients will also be administered a second therapeutic agent or a chemotherapy regimen in addition to Compound C. In some embodiments, the second therapeutic agent is nivolumab, which is administered at specified intervals. In some embodiments, the chemotherapy regimen is FOLFIRI or Gem/ABRAXANE (nab-paclitaxel), which is administered at specified intervals.

### Study Design

The study is conducted in 2 parts. Part 1 will evaluate safety, tolerability, PK, and PD of two different doses of Compound C (i.e., 300 mg BID or 600 mg QD) in combination with either FOLFIRI (Arm A), Gem/ABRAXANE (nab-paclitaxel) [Arm B], or nivolumab (Arm C) in patients with advanced colorectal and pancreatic cancers. Part 2 is a dose expansion study to assess preliminary efficacy of Compound C in combination with either chemotherapy or nivolumab in patients with advanced colorectal or pancreatic cancers. Arm D (Compound C monotherapy) will open if participants in Arm C show an objective response rate (ORR) of approximately 15% or durable responses are seen with the combination of nivolumab and Compound C.

The objectives and endpoints for the primary and secondary analyses of this study are shown in Table 3 (Part 1) and Table 4 (Part 2).

TABLE 3
Objectives and Endpoints (Part 1)

| Objectives | Endpoints |
|---|---|
| Primary | |
| 1) To assess the safety and tolerability of Compound C in | 1a) Incidence of AEs, SAEs, AEs meeting |
| combination with either FOLFIRI (Arm A), Gem + | protocol-defined DLT criteria, AEs |
| nab-paclitaxel (Arm B), or nivolumab (Arm C) in | leading to discontinuation, and death; |
| participants with advanced CRC or pancreatic cancer | Incidence of laboratory abnormalities |
| | 1b) Summary measures of vital signs or ECGs |
| Secondary | |
| 1) To assess the preliminary efficacy of Compound C in | 1) Overall Response Rate, Median |
| combination with either FOLFIRI (Arm A), Gem + | Duration of Response and Progression |
| nab-paclitaxel (Arm B), or nivolumab (Arm C) in | Free Survival Rate at 24 weeks |
| participants with advanced CRC or pancreatic cancer | 2a) PK parameters, such as Cmax, Tmax, |
| 2) To characterize the PK of Compound C and its | Ctrough, Ctau, AUC (0-8), AUC (TAU), |
| metabolite when administered alone, and in | CLT/F, AT, CLR, % UR, MR_Cmax, and |
| combination with either Gem + nab-paclitaxel, | MR_AUC (TAU), if data permit |
| FOLFIRI or nivolumab | 2b) Cmax and Ctrough concentrations of |
| 3) To characterize the immunogenicity of nivolumab when | Compound C during combination therapy |
| administered in combination with Compound C | 3) Frequency of positive anti-drug antibody |
| 4) To assess the pharmacodynamic effects | (ADA) to nivolumab during |
| of Compound C in tumor samples | combination therapy |
| | 4) Decrease in Treg & TAM in tumor samples |

Abbreviations: %UR = percent urinary recovery over dosing interval;
ADA = anti-drug antibody;
AEs = adverse events;
AT = accumulation index;
AUC (0-8) = area under the concentration-time curve from time 0 to 8 hours post dose;
AUC (TAU) = area under the concentration-time curve in 1 dosing interval;
CCR2 = cysteine-cysteine chemokine receptor 2;
CL = clearance;
CLR = renal clearance;
CLT/F = apparent total body clearance;
Cmax = maximum observed plasma concentration;
CRC = colorectal cancer;
Ctau = observed plasma concentration at the end of the dosing interval;
Ctrough = trough observed plasma concentration;
DLT = dose limiting toxicity;
ECGs = electrocardiograms;
Gem = gemcitabine;
MATE = multidrug and toxin extrusion protein;
MCP-1 = monocyte chemotactic protein-1;
MR_AUC (TAU) = ratio of metabolite AUC (TAU) to parent AUC (TAU), corrected for molecular weight;
MR_Cmax = ratio of metabolite Cmax to parent Cmax, corrected for molecular weight;
NMN = N-methylnicotinamide;
OS = overall survival;
PK = pharmacokinetic(s);
SAEs = serious adverse events;
TAM = tumor-associated macrophages;
Tmax = time of maximum observed plasma concentration;
Treg = regulatory T cells

TABLE 4
Objectives and Endpoints (Part 2)

| Objectives | Endpoints |
|---|---|
| Primary | |
| 1) To assess the preliminary efficacy of Compound C in | 1) Overall Response Rate, Median |
| combination with either FOLFIRI (Arm A), Gem + | Duration of Response, and Progression |
| nab-paclitaxel (Arm B), or nivolumab (Arm C) in | Free Survival Rate at 24 weeks |
| participants with advanced CRC or pancreatic cancer | |
| Secondary | |
| 1) To assess the safety and tolerability of Compound C in | 1a) Incidence of AEs, SAEs, AEs leading to |
| combination with either FOLFIRI (Arm A), Gem + | discontinuation, and death; Incidence of |
| nab-paclitaxel (Arm B) and nivolumab (Arm C) in | laboratory abnormalities |
| participants with advanced CRC or pancreatic cancer | 1b) Summary measures of vital signs or ECG |
| 2) To assess the pharmacodynamic effects of Compound | 2) Decrease in Treg & TAM in tumor samples |
| C in tumor samples | |

Abbreviations: ADA = anti-drug antibody;
AEs = adverse events;
Gem = gemcitabine;
SAEs = serious adverse events;
Treg = regulatory T cells

### Study Population

### Inclusion Criteria (Study Open to all Sexes, 18 Years and Older):

Participants must have metastatic colorectal or pancreatic cancer
Eastern Cooperative Oncology Group (ECOG) performance status of <1
Ability to swallow pills or capsules
All participants will be required to undergo mandatory pre and on-treatment biopsies
Adequate marrow function
Adequate other organ functions
Ability to comply with study visits, treatment, procedures, PK and PD sample collection, and required study follow-up

### Exclusion Criteria:

Histology other than adenocarcinoma (neuroendocrine or acinar cell)
Suspected, known, or progressive CNS metastases (Imaging required only if participants are symptomatic)
Participants with active, known or suspected autoimmune disease
Participants with a condition requiring systemic treatment with either corticosteroids (>10 mg daily prednisone equivalents) or other immunosuppressive medications within 14 days of study treatment administration
Interstitial lung disease that is symptomatic or may interfere with the detection or management of suspected treatment-related pulmonary toxicity
Prior treatment with CCR2 and/or CCR5 inhibitors
History of allergy to study treatments or any of its components of the study arm that participant is enrolling

### Treatment Period (Part 1)

Treatment period (Part 1) will have a 2-week monotherapy lead-in with Compound C prior to combination with either FOLFIRI, Gem/nab-paclitaxel, or nivolumab. All three arms will enroll participants in parallel. Participants will be assigned to the Compound C 300 mg BID or 600 mg QD cohort in each arm of the study. Approximately 6 evaluable participants will be treated at each Compound C dose (i.e., 300 mg BID or 600 mg QD) for a total of approximately 12 participants per arm. Up to 6 additional participants may be added to a dose cohort to better characterize the safety, PK, or PD profile and inform Part 2 dose selection of Compound C if needed after discussion with the Sponsor and investigators.

The Compound C monotherapy lead-in allows assessment of initial tolerability in cancer participants, facilitating the characterization of the added or synergistic toxicity of the subsequent combination regimens, and enables a biopsy at 2 weeks to characterize PD effects of Compound C. After 2 weeks of Compound C monotherapy, participants will start the combination phase with either FOLFIRI, Gem/nab-paclitaxel, or nivolumab along with continued treatment with Compound C after a mandatory biopsy (±3 days) is performed. Monotherapy should continue if biopsy is collected beyond 2 weeks and combination with chemotherapy or nivolumab should only begin after the biopsy is performed. Participants will continue on combination until disease progression, intolerance, meeting criteria for treatment discontinuation, or withdrawal of consent.

### Treatment Period (Part 2)

Treatment period (Part 2) will explore the preliminary signals of efficacy of Compound C with various combinations as described below. Part 2 will open to enrollment once a Compound C dose and schedule has been selected from the doses being investigated in Part 1. The dose of Compound C in Part 2 will be chosen based on safety, PK, and PD data available from Part 1 of the study, and will not exceed dose and schedule determined to be safe in Part 1. Participants in Part 2 will be treated with Compound C in combination with either FOLFIRI, Gem/nab-paclitaxel, or nivolumab without a Compound C monotherapy lead-in.

Initially, there will be three study arms (Arms A, B, and C) containing a total of 6 cohorts (30 to 40 evaluable participants in each cohort). Arm D (Compound C monotherapy) will open if participants in Arm C show an objective response rate (ORR) of approximately 15% or durable responses are seen with the combination of nivolumab and Compound C.

A biopsy will be obtained 4 weeks (±3 days) after the first dose in Part 2 for correlative studies. Participants on bevacizumab in Arm A should be off bevacizumab for at least 14 days or according to institutional guidelines to prevent any bleeding or delay in wound healing.

Participants will continue on combination until disease progression, intolerance, meeting criteria for treatment discontinuation, or withdrawal of consent.

### Treatment Period (Parts 1 and 2)

Blood, urine, stool, and tumor biopsy samples and electrocardiograms (ECGs) will be collected and participants will receive study treatments as per the schedule of activities. Participants will have baseline imaging within approximately 28 days of start of the study and then every 8 weeks after starting combination treatment for reassessment. Tumor progression or response endpoints will be assessed using RECIST v1.1 for solid tumors. Participants will continue on treatment until disease progression, clinical deterioration, toxicity, meeting criteria for discontinuation of study treatment, or withdrawal of consent. Participants who go off treatment will be followed for safety assessments and survival status.

Participants with a response of SD, PR, or CR at the end of a given cycle will continue to the next treatment cycle. Participants will generally be allowed to continue study treatment until the first occurrence of either 1) PD, 2) clinical deterioration suggesting that no further benefit from treatment is likely, 3) intolerability to therapy, 4) the participant meets criteria for discontinuation of study treatment.

Physical examinations, vital sign measurements, 12-lead electrocardiograms (ECG), and clinical laboratory evaluations will be performed at selected times throughout the dosing interval. In the event of multiple procedures are required at a single time point, the following is a list of procedures from highest priority to low: PK sampling, ECG and vital signs, and laboratory tests. Participants will be closely monitored for AEs throughout the study. Blood and urine samples will be collected at baseline and after study treatment administration for PK and multidrug and toxin extrusion (MATE)-renal transporter biomarker analyses according to the schedules for pharmacokinetic studies.

### Treatment Arms and Duration:

TABLE 5
Study Treatment:

| Medication | Potency |
|---|---|
| Compound C Capsule | 150 mg |
| Nivolumab Injection^{a} | 100 mg/vial (10 mg/mL) |
| Nivolumab Injection^{a} | 40 mg/vial (10 mg/mL) |
| Gemcitabine Injection^{b} | 1000 mg/vial and various |
| | strengths |
| Nab-paclitaxel | 100 mg/vial and various |
| (ABRAXANE)^{b} | strengths |
| 5-FU^{b} | Various strengths |
| Leucovorin^{b} | Various strengths |
| Irinotecan^{b} | Various strengths |

^{a}Nivolumab will be supplied as a 240 mg kit-each kit containing (2) 100 mg vials and (1) 40 mg vial
^{b}These products will be obtained as local commercial product in countries if allowed by local regulations or through investigating site’s standard prescribing procedures, otherwise the Sponsor will supply these products.

TABLE 6
Selection and Timing of Dose

| | | Frequency of | Route of |
|---|---|---|---|
| Study Treatment | Dosage level(s) | Administration | Administration |
| Compound C | 300 mg | BID | PO |
| (150 mg capsule) | | | |
| Compound C | 600 mg | QD | PO |
| (150 mg capsule) | | | |
| Compound C | 600 mg | BID^{a} | PO |
| (150 mg capsule) | | | |
| Nivolumab | 480 mg IV | Q4W | IV infusion |
| 5-FU | 400 mg/m² | Day 1, 15: Q4W | Bolus and |
| | Bolus AND | | IV infusion |
| | 2400 mg/m² IV | | |
| Leucovorin | 400 mg/m² IV | Day 1, 15: Q4W | IV infusion |
| Irinotecan | 180 mg/m² IV | Day 1, 15: Q4W | IV infusion |
| Gemcitabine | 1000 mg/m² IV | Day 1, 8, 15: Q4W | IV infusion |
| Nab-paclitaxel | 125 mg/m² IV | Day 1, 8, 15: Q4W | IV infusion |
| (ABRAXANE) | | | |

Abbreviations: 5-FU =5-fluorouracil;
BID = twice a day;
IV = intravenous;
PO = per os (by mouth [orally]);
Q4W = every 4 weeks;
QD = once daily
^{a}The 600 mg BID regimen may be investigated to explore Compound C PK/PD relationships for potential dose optimization (See Section 5.5.1)

FOLFIRI (irinotecan 180 mg/m² on Day 1 over 90 minutes; leucovorin 400 mg/m² over 2 hours on Day 1 (leucovorin may be given concurrently with irinotecan); 5-FU 400 mg/m² bolus on Day 1, followed by 2400 mg/m² over 46 hours continuous infusion) on Days 1 and 15 of a 28-day cycle. Bevacizumab, cetuximab, or panitumumab can be added to 1 L FOLFIRI if appropriate, and will be administered in accordance with local Health Authority approved labeling for these agents. Levoleucovorin can be substituted for leucovorin or flat dose of leucovorin can be used as per site's standard practice.

The recommended dose of nab-paclitaxel (ABRAXANE) is 125 mg/m² administered as an intravenous infusion over 30 to 40 minutes on Days 1, 8, and 15 of each 28-day cycle. Administer gemcitabine 1000 mg/m² over 30 to 40 minutes immediately after nab-paclitaxel on Days 1, 8, and 15 of each 28-day cycle.

Nivolumab 480 mg administered as an intravenous infusion over 30 minutes every 4 weeks.

Treatment Duration: Participants will be treated until disease progression, intolerance to treatment, meeting discontinuation criteria, or withdrawal of consent. Participants may be treated beyond progression as long as they meet the criteria in Section 7.4.8. Participants who discontinue chemotherapy in part or whole due to intolerance can continue Compound C on study after investigator's discussion with the Medical Monitor or Sponsor designee. Participants will continue to get all study evaluations as per schedule of events in for study assessments and procedures for on-treatment in different arms.

### Efficacy Assessments

Disease assessment with computed tomography (CT) and/or MRI, as appropriate, will be performed at baseline and approximately every 8 weeks (±1 week from Cycle 1 Day 1) until disease progression, treatment discontinuation, withdrawal from study, or start of subsequent treatment, whichever is earlier.

### Imaging Assessment for the Study

Disease Tumor assessment with contrast-enhanced CT scans acquired on dedicated CT equipment is preferred for this study. Contrast-enhanced CT of the chest, abdomen, pelvis, and other known/suspected sites of disease should be performed for tumor assessments. Should a participant have contraindication for CT intravenous contrast, a non-contrast CT of the chest and a contrast-enhanced MRI of the abdomen, pelvis, and other known/suspected sites of disease should be obtained.

Should a participant have contraindication for both MR and CT intravenous contrasts, a non-contrast CT of the chest and a non-contrast MRI of the abdomen, pelvis, and other known/suspected sites of disease should be obtained.

Should a participant have contraindication for MRI (eg, incompatible pacemaker) in addition to contraindication to CT intravenous contrast, a non-contrast CT of the chest, abdomen, pelvis, and other known/suspected sites of disease is acceptable.

CT and MRI scans should be acquired with slice thickness of 5 mm or less with no intervening gap (contiguous). Every attempt should be made to image each participant using an identical acquisition protocol for all imaging time points.

Use of CT component of a positron emission tomography (PET)-CT scanner: Combined modality scanning such as with fluorodeoxyglucose (FDG) PET-CT is increasingly used in clinical care, and is a modality/technology that is in rapid evolution; therefore, the recommendations outlined here may change rather quickly with time. At present, low dose or attenuation correction CT portions of a combined FDG PET-CT are of limited use in anatomically-based efficacy assessments and it is therefore suggested that they should not be substituted for dedicated diagnostic contrast enhanced CT scans for anatomically-based RECIST measurements. However, if a site can document that the CT performed as part of a FDG PET-CT is of identical diagnostic quality to a diagnostic CT (with IV and oral contrast) then the CT portion of the FDG PET-CT can be used for RECIST 1.1 measurements. Note, however, that the FDG PET portion of the CT introduces additional data which may bias an investigator if it is not routinely or serially performed.

Participants with a history of bone metastasis may have a bone scan, if clinically indicated.

Assessments will be performed at baseline and at the time points described per RECIST v1.1 criteria, until disease progression per RECIST v1.1 criteria, or withdrawal from the study.

What is claimed is:

1. A method of treating cancer in a subject comprising administering to the subject a combination of a monoclonal antibody, a compound of Formula (I), or a pharmaceutically acceptable salt thereof, or a combination thereof, 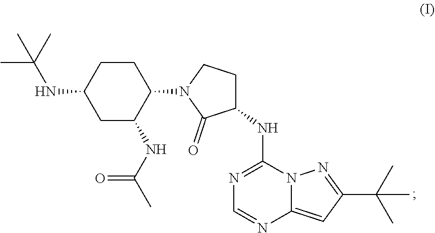 [SMILES: CC(=O)N[C@@H]1C[C@H](NC(C)(C)C)CC[C@@H]1N1CC[C@H](Nc2ncnc3cc(C(C)(C)C)nn23)C1=O]
and/or chemotherapy.

2. A method of treating cancer in a subject comprising administering to the subject a combination of a monoclonal antibody and a compound of Formula (I), or a pharmaceutically acceptable salt thereof, or a combination thereof,

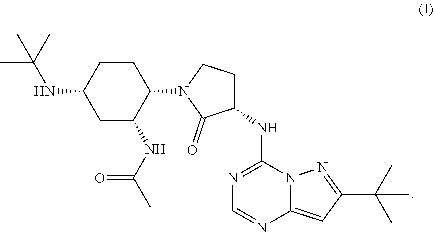 [SMILES: CC(=O)N[C@@H]1C[C@H](NC(C)(C)C)CC[C@@H]1N1CC[C@H](Nc2ncnc3cc(C(C)(C)C)nn23)C1=O]

3. The method of claim 1, wherein the amount of the compound of Formula (I) administered to the subject is from about 25 mg per day to about 1200 mg per day.

4. The method of claim 3, wherein the amount of the compound of Formula (I) administered to the subject is from about 100 mg per day to about 1200 mg per day.

5. The method of claim 4, wherein the amount of the compound of Formula (I) administered to the subject is from about 200 mg per day to about 1200 mg per day.

6. The method of claim 5, wherein the amount of the compound of Formula (I) administered to the subject is from about 300 mg per day to about 1200 mg per day.

7. The method of claim 6, wherein the amount of the compound of Formula (I) administered to the subject is from about 600 mg per day to about 1200 mg per day.

8. The method of claim 3, wherein the amount of the compound of Formula (I) administered to the subject is from about 25 mg per day to about 600 mg per day.

9. The method of claim 8, wherein the amount of the compound of Formula (I) administered to the subject is from about 25 mg per day to about 600 mg per day.

10. The method of claim 9, wherein the amount of the compound of Formula (I) administered to the subject is from about 100 mg per day to about 600 mg per day.

11. The method of claim 10, wherein the amount of the compound of Formula (I) administered to the subject is from about 200 mg per day to about 600 mg per day.

12. The method of claim 11, wherein the amount of the compound of Formula (I) administered to the subject is from about 300 mg per day to about 600 mg per day.

13. The method of claim 1, wherein the amount of the compound of Formula (I) administered in doses of 300, and 600 mg either once or twice a day.

14. The method of claim 1, wherein the cancer is a malignant solid tumor.

15. The method of claim 14, wherein the cancer is metastatic and/or unresectable.

16. The method of claim 15, wherein the cancer is selected from colorectal cancer, pancreatic cancer, liver cancer and lung cancer, or a combination thereof.

17. The method of claim 16, wherein the cancer is colorectal cancer or pancreatic cancer.

18. The method of claim 17, wherein the subject has received at least one prior therapy for the treatment of the cancer.

19. The method of claim 17, wherein the subject is treatment naïve.

20. The method of claim 19, wherein the monoclonal antibody is nivolumab.

21. The method of claim 20, wherein the nivolumab is administered by intravenous infusion at a dose of about 480 mg every 4 weeks.

22. The method of claim 21, wherein the chemotherapy is FOLFIRI or Gem/ABRAXANE.


SEQUENCE LISTING (2 sequences)

SEQ ID NO: 1 - protein, 216 aa, Artificial Sequence
  Antibody B Light chain variable
DTVLTQSPALAVSLGQRVTISCKASETVSSSMYSYIHWYQQKPGQQPKLLIYRASNLESGVPARFSGSGSGTDFTLTIDPVEADDVATYFCQQSWNPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 2 - protein, 445 aa, Artificial Sequence
  Antibody B heavy chain variable
QVQLKESGPGLVQPSQTLSLTCTVSGFSLTSYNVHWVRQPPGKGLEWMGGMRYNEDTSYNSALKSRLSISRDTSKNQVFLKMNSLQTDDTGTYYCTRDAVYGGYGGWFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK